(19) United States
(12) Patent Application Publication
(10) Pub. No.: US 2025/0290026 A1
(43) Pub. Date: Sep. 18, 2025

(54) TISSUE PROCESSING SYSTEM AND METHOD OF USE THEREOF

(71) Applicants: Mihail VALERIEV, Sofia (BG); Randolph BEIMEL, Boca Raton, FL (US)

(72) Inventors: Marzia DELUCA, Milano (IT); Carlo VENTURA, Bologna (IT); Luca VIGNOLA, Verona (IT)

(21) Appl. No.: 19/221,942

(22) Filed: May 29, 2025

Related U.S. Application Data
(63) Continuation-in-part of application No. PCT/IL2023/051234, filed on Dec. 1, 2023.
(60) Provisional application No. 63/429,588, filed on Dec. 2, 2022.
(60) Provisional application No. 63/656,315, filed on Jun. 5, 2024.

(51) Int. Cl. C12M 1/00; A61K 8/98; C12M 1/26; C12N 5/077

(52) U.S. Cl. CPC C12M 47/04; A61K 8/981; C12M 23/14; C12M 29/20; C12M 33/14; C12N 5/0653

(57) ABSTRACT

The present invention pertains to systems and methods of processing a biological tissue from a subject. Particularly, the invention provides a tissue processing system for processing an extracted biological tissue, comprising tissue processing chamber comprising an expandable bag and configured for cleaning and separation of components in the extracted tissue; at least one inlet, wherein the at least one inlet is for administering an extracted biological tissue into the tissue processing chamber; and a waste bag for collection of water-soluble components separated from the extracted tissue.

### CROSS REFERENCE TO RELATED APPLICATIONS

This application is a Continuation in Part of PCT Patent Application No. PCT/IL2023/051234 having International filing date of Dec. 1, 2023, which claims the benefit of priority of U.S. Provisional Patent Application No. 63/429,588, filed Dec. 2, 2022. This application also claims the benefit of priority of U.S. Provisional Patent Application No. 63/656,315, filed Jun. 5, 2024. The contents of the aforementioned applications are all incorporated herein by reference in their entirety.

### FIELD OF THE INVENTION

The present invention relates to a tissue processing system and method of use thereof. Moreover, the present invention is of a system for processing adipose tissue for autologous use.

### BACKGROUND OF THE INVENTION

Adipose tissue as a lipofiller finds widespread use in aesthetics and for tissue regeneration in fields such as orthopedics, gynecology, general surgery, and urology. Micro-fragmented adipose tissue is used in orthopedics to provide mechanical support and promote healing.

Adipose tissue is a good source of stem cells. The stem cells are concentrated in the vascular-stromal niche. Adipose tissue liposuctioned from a patient must be processed before reuse. The adipose cells must be separated from other material such as blood, cell debris, oil from ruptured cells and the cells must be preferably declustered. Common methods of separation of the components in the liposuctioned tissue involve centrifugation, emulsification, mincing, washing and precipitation. However, these methods suffer from disadvantages, such as non-optimally removing cell fragments and causing a significant number of adipocytes to break, as well as loss of important biological components. The methods of multi fragmentation are multi step, use operator dependent mechanical compression and are not closed sterile systems.

It would be desirable to have a system for processing adipose tissue, which is a closed sterile system for providing sterile purified adipose tissue. It would be advantageous if the system would eliminate inflammation promoting components from the adipose tissue. It would be beneficial if the system reduced adipose clusters and preserved the intact vascular-stromal niche. It would also be useful to have a method, which is facile giving reproducible results. The present invention provides such a system and method of use thereof.

### SUMMARY

The invention may have several aspects. In one aspect the invention provides a tissue processing system for processing an extracted biological tissue, such as an adipose tissue of a subject, the system comprises a tubing system, a tissue processing chamber comprising an expandable bag, at least one inlet for introducing a physiological solution and an extracted tissue, at least one outlet for collecting waste and the processed tissue, and at least one filter.

The system and method provide the following attributes: preparation of readily available Unified Fluid Adipose Tissue (UFAT) derivative recapitulating within a single product all the traits that compose the biophysical properties of an original fat tissue, closed circuit from sampling to processing throughout the processing chain; total absence of air, no risk of oxidation with complete priming of the system; processing in physiological solution, cluster reduction without cell trauma; tissue washing, tissue micro-sizing and/or nano-sizing; tissue fragmentation with plastic filters; purification of pro-inflammatory elements; automatic non-operator dependent loading; volume control at every stage of processing; implementable waste bag; waste bag with outputs for cell recovery; total removal of blood residues; no centrifugation and no enzymes or other additives and manipulations are required.

In one embodiment, the system makes no use of any enzymes. In one or more embodiments, the method does not use enzymes for tissue processing. In one embodiment, the system makes no use of centrifugation. In one or more embodiments, the method does not use centrifuge the tissue. In one embodiment, the system makes no use of any pump or other electronic or mechanical pumping devices. In one or more embodiments, the method does not pump the tissue utilizing a mechanical or electrical device except for the expandable bag which passively contacts and expels the solution and/or sample when the outlet is opened. In one or more embodiments, the filter is outside of the expandable bag. In one or more embodiment, the filter is outside of the tissue processing chamber.

In one or more embodiments, the tissue processing chamber comprises an expandable bag disposed within the housing that comprises a movable piston that surrounds central rod, wherein when the expandable bag is filled with tissue sample and/or physiological fluid, the expandable bag is expanded to various directions, and the piston is pushed downwardly. In one or more embodiments, the housing comprises a piston that is movable in response to expansion of the expandable bag when filled with the extracted adipose tissue and physiological solution.

The herein processed tissue advantageously showed an increase in the number of MUSE cells after 24 hours at 4° C. This indicates that the MUSE cell population is well-preserved. Thus, when the processed and cooled down tissue is injected within a damaged tissue, all the hostile environment encountered by the MUSE cells, including hypoxia, ischemia, oxidative stress, inflammation, senescence, act as a major stressor to trigger the MUSE cells themselves out of their dormant state, maximizing their rescuing potential.

Optionally, the herein tissue product is cryopreserved (to below 0° C., e.g., to −10° C. or below, or to −20° C. or below, optionally using liquid nitrogen) before being injected. That is, the herein processed tissue can be subjected to cryopreservation, while illustrating the unique advantage that the tissue maintains the MUSE cells in an active pro-regenerative state, readily amenable after thawing and subsequent injection of the product itself.

In one or more embodiments, the herein processed tissue advantageously comprises MUSE cells, i.e., even following the processing stages. Moreover, exposing the processed tissue to a temperature induced stress at 4° C. for 24 h elicited a remarkable increase in the number of MUSE cells. Thus, in one or more embodiments, after the processing stage as described herein there may be an additional step of eliciting stress, such as cooling down the processed tissue before injection thereof to the subject.

The cooling may be for any temperature below room temperature, for example to a temperature of below 25° C., below 20° C., below 15° C., below 10° C., below 5° C., or below 0° C. For example, the temperature may be in a range of 0° C.-10° C., or 0° C.-8° C., 0° C.-6° C., 0° C.-4° C., 2° C.-6° C. Each possibility represents a separate embodiment of the invention. The cooling may be for a period of a few hours or a few days, e.g., for 10, 12,24, or 48 hours or any value in between. Each possibility represents a separate embodiment of the invention.

In one aspect the invention provides a tissue processing system for processing an extracted biological tissue, comprising:
a tissue processing chamber comprising an expandable bag and configured for cleaning, separation and/or fragmentation of components in the extracted tissue; and
at least one inlet, wherein the at least one inlet is for administering an extracted biological tissue into the tissue processing chamber.

In one aspect the invention provides a tissue processing system for processing extracted adipose tissue, comprising:
a tissue processing chamber comprising an expandable bag configured for holding fluid and the adipose tissue, the tissue processing chamber is further configured for cleaning, separation, and/or fragmentation of components in the extracted tissue, the expandable bag is constructed from an elastic material, such that the bag expands when fluid is introduced into the bag and passively contracts when the fluid is removed from the bag, the expandable bag applies radial compression forces on the fluids inside thereby affording a controllable fluid flow rate out of the chamber,
at least one outlet for collecting waste and processed tissue;

at least one inlet for administering an extracted biological tissue into the tissue processing chamber; and
a waste container for collection of water-soluble components separated from the extracted tissue.

In one or more embodiments, the system further includes a waste container for collection of water-soluble components separated from the extracted tissue. In one or more embodiments, the waste container is selected from a waste bag, a bottle, and a tube. In one or more embodiments, the waste container is a waste bag.

In a further aspect the invention provides a tissue processing system for processing an extracted biological tissue, comprising:
a tissue processing chamber comprising an expandable bag and configured for cleaning and separation of components in the extracted tissue;
at least one inlet, wherein the at least one inlet is for administering an extracted biological tissue into the tissue processing chamber; and
a waste bag for collection of water-soluble components separated from the extracted tissue.

In one or more embodiments, the expandable bag is disposed within a housing that comprises a movable piston that surrounds a central rod, wherein when the expandable bag is filled with tissue sample and/or physiological fluid, the expandable bag is expanded to various directions, and the piston is pushed downwardly.

In one or more embodiments, the system comprises at least one inlet. In one or more embodiments, the system comprises at least two inlets. In one or more embodiments, the system comprises at least three inlets. In one or more embodiments, the system comprises one inlet. In one or more embodiments, the system comprises two inlets. In one or more embodiments, the system comprises three inlets.

In one or more embodiments, the system comprises at least one outlet. In one or more embodiments, the system comprises at least two outlets. In one or more embodiments, the system comprises at least three outlets. In one or more embodiments, the system comprises one outlet. In one or more embodiments, the system comprises two outlets. In one or more embodiments, the system comprises three outlets.

In one or more embodiments, the extracted tissue is an adipose tissue. In one or more embodiments, the adipose tissue is extracted from an adipose depot.

In one or more embodiments, the system further comprising at least one channel. In one or more embodiments, the at least one channel is used for providing a physiological solution into the system. In one or more embodiments, the system comprises a purging apparatus comprising at least one channel and the purging apparatus is used for providing a physiological solution into the system.

In one or more embodiments, the at least one channel is opened and closed with a valve. In one or more embodiments, the valve is a one-way valve. In one or more embodiments, the valve is a multi-way valve. In one or more embodiments, the valve is a three way valve. In one or more embodiments, the channel comprises tubing connected to an inlet of the system.

In one or more embodiments, the system comprises at least one inlet. In one or more embodiments, the system comprises a plurality of inlets. In one or more embodiments, the system comprises at least two inlets. In one or more embodiments, the system comprises at least three inlets.

In one or more embodiments, the system comprises at least one outlet. In one or more embodiments, the system comprises a plurality of outlets. In one or more embodiments, the system comprises at least two outlets. In one or more embodiments, the system comprises at least three outlets.

In one or more embodiments, the physiological solution is for washing the system. In one or more embodiments, the physiological solution is for washing the tissue. In one or more embodiments, the physiological solution is selected from the group consisting of saline, Phosphate Buffer Saline, water, and cells' culture media.

In one or more embodiments, the system further comprising at least one filter. In one or more embodiments, the at least one filter is having pore size of about 1200 microns (μm). In one or more embodiments, the at least one filter is having pore size of about 700 microns (μm). In one or more embodiments, the at least one filter is having pore size of about 1200 microns (μm) or below. In one or more embodiments, the at least one filter is having pore size of about 700 microns (μm) or below. In one or more embodiments, the at least one filter is having pores size of about 600 microns (μm) or above. In one or more embodiments, the at least one filter is having pores size of about 700 microns (μm) or above.

In one or more embodiments, the at least one filter is connected to an inlet of the at least one inlets and is configured to allow administration of the extracted tissue into the system via the at least one filter.

In one or more embodiments, the tissue processing chamber comprises at least one outlet. In one or more embodiments, the tissue processing chamber comprises at least one inlet.

In one or more embodiments, the tissue processing chamber comprises at least one inlet and at least one outlet. In one or more embodiments, the tissue processing chamber comprises an expandable bag within a housing.

In one or more embodiments, the waste bag is connected to an outlet of the at least one outlet of the tissue processing chamber via a channel. In one or more embodiments, the channel is opened and closed with a valve.

In one or more embodiments, the expandable bag is configured for holding adipose tissue. In one or more embodiments, the expandable bag is configured for holding an aqueous physiological solution. In one or more embodiments, the expandable bag is configured for holding adipose tissue and an aqueous physiological solution.

In one or more embodiments, the expandable bag is manufactured from a rubber material having visco-elastic properties, or from a polymer characterized by elastic properties.

In one or more embodiments, the system does not include a centrifuge, or an electric pump and/or does not require the use of an enzyme for processing the tissue.

In one or more embodiments, an outlet of the at least one outlet is connected to a syringe port for extraction of the purified/processed tissue.

In one or more embodiments, the waste bag comprises at least one outlet port for extracting/harvesting at least one component held in the waste bag.

The present invention further provides a method of processing a tissue comprising:
providing a tissue processing system;
administering a physiological aqueous solution into an expandable bag of a tissue processing chamber within the tissue processing system;
administering adipose tissue extracted/harvested from a subject into a tissue processing chamber, the tissue administered via an inlet of the system into an expandable bag of the tissue processing chamber;
shaking the contents of the tissue processing chamber;
removing an aqueous phase from the tissue processing chamber into a waste container; and
removing the purified/processed adipose tissue out of the tissue processing chamber and out of the processing system.

The present invention further provides a method of processing a tissue comprising:
providing a tissue processing system;
administering a physiological aqueous solution into an expandable bag of a tissue processing chamber within the tissue processing system, the expandable bag is constructed from an elastic material, such that the bag expands when fluid is introduced into the bag and passively contracts when the fluid is removed from the bag;
administering adipose tissue extracted from a subject into the tissue processing
shaking the contents of the tissue processing chamber;
removing the aqueous phase from the tissue processing chamber into a waste container; and
removing the purified adipose tissue out of the tissue processing chamber and out of the tissue processing system, wherein the administering and removing steps are conducted manually.

In one or more embodiments, prior to administering the adipose tissue into the system, the method comprises purging the system with a physiological aqueous solution.

In one or more embodiments, prior to shaking the contents of the tissue processing chamber, the method comprises reversing the tissue processing chamber upside down.

In one or more embodiments, the system comprises at least one channel for providing a physiological solution to the system, wherein access of the channel to the system is opened and closed with a multi way valve, and wherein the purging comprises opening the multi way valve to provide the physiological solution into the system.

In one or more embodiments, the method further comprising purging the tissue processing chamber with a physiological aqueous solution, wherein the system comprises at least one channel for providing a physiological solution to the tissue processing chamber, wherein access of the channel to the tissue processing chamber is opened and closed with a multi way valve, and wherein the purging comprises opening the multi way valve to provide the physiological solution into the tissue processing chamber.

In one or more embodiments, the purging comprises removing air bubbles.

In one or more embodiments, the administering adipose tissue into the system comprises administering the adipose tissue through a filter connected to the system, the filter disposed prior to the inlet of the tissue processing chamber and the method further comprises filtering the adipose tissue.

In one or more embodiments, the filter is a having pore size of at least about 70 microns (μm). In one or more embodiments, the filter is a having pore size of at least about 70 microns (μm) and up to about 3000 microns (μm). In one or more embodiments, the filter is a having pore size of at least about 700 microns (μm). In one or more embodiments, the filter is a having pore size of at least about 700 microns (μm) and up to about 2400 microns (μm).

In one or more embodiments, the method comprises:
administering a physiological aqueous solution into the expandable bag of the tissue processing chamber;
administering the adipose tissue through a first filter connected to the system, the filter is optionally having pores size of about 1200 microns (μm) and disposed prior to the inlet of the tissue processing chamber and filtering the adipose tissue into the expandable bag;
shaking the contents of the tissue processing chamber;
removing the aqueous phase from the tissue processing chamber into the waste bag; and
removing the purified adipose tissue out of the tissue processing chamber and out of the processing system.

In one or more embodiments, the method comprises:
administering a physiological aqueous solution into the expandable bag of the tissue processing chamber;
administering the adipose tissue through a second filter connected to the system, the second filter has pores size that is smaller than the pore size of the first filter, the second filter is optionally of at least about 700 microns (um) and disposed prior to the inlet of the tissue processing chamber and filtering the adipose tissue into the expandable bag;
optionally reversing the tissue processing chamber;
shaking the contents of the tissue processing chamber;
removing the aqueous phase from the tissue processing chamber into the waste bag; and
removing the purified adipose tissue out of the tissue processing chamber and out of the processing system.

In one or more embodiments, the administering the extracted tissue into an expandable bag of the tissue processing chamber is conducted prior to or after the administering an aqueous solution into the expandable bag of the tissue processing chamber.

In one or more embodiments, the administering an aqueous solution comprises administering about the same volume of aqueous solution as the volume of extracted tissue administered to the tissue processing chamber.

In one or more embodiments, the shaking comprises manual or mechanical shaking for solubilizing water soluble components in the aqueous phase.

In one or more embodiments, after shaking and stabilizing the tissue processing chamber, a two-phase solution is formed.

In one or more embodiments, the waste bag is connected to an outlet of the tissue processing chamber via a channel and wherein the channel is opened and closed with a multi way valve and wherein removing the aqueous phase from the tissue processing chamber into the waste bag comprises opening the multi way valve to allow the aqueous fluid to flow through the channel from the tissue processing chamber and into the waste bag.

In one or more embodiments, after the removing the aqueous phase from the tissue processing chamber into the waste bag, the method further comprising repeating the steps of
administering an aqueous solution into the expandable bag of the tissue processing chamber;
shaking the contents of the tissue processing chamber; and
removing the aqueous phase from the tissue processing chamber into the waste bag.

In one or more embodiments, removing the purified adipose tissue out of the system comprises extracting the tissue with a syringe via an outlet in the system.

In one or more embodiments, the extracted purified adipose tissue is administered to the same subject it was extracted from.

In one or more embodiments, the extracted purified adipose tissue is for a cosmetic or medical treatment of the subject.

In one or more embodiments, the extracted purified adipose tissue is administered to a different subject from the subject it was extracted from.

In one or more embodiments, the extracted purified adipose tissue is used in a formulation for a cosmetic or a medical treatment.

In one or more embodiments, the aqueous phase comprises physiological fluids, exosomes, and inflammatory agents, and wherein the method further comprising separating the exosomes from the waste bag and collecting them.

The present invention further provides a tissue sample prepared by the herein method.

The present invention further provides a method of treating a subject comprising obtaining a tissue sample according to the herein method and administering the tissue sample to the subject.

The present invention further provides a use of the herein processed tissue sample in the treatment of a therapeutic indication.

In one or more embodiments, the method further comprising cooling down the processed tissue to a temperature of less than 4° C. prior to injection thereof to the subject.

In one or more embodiments, the subject is a mammal. In one or more embodiments, the mammal is a human. In one or more embodiments, treating comprises a cosmetic indication, an orthopedic indication (e.g., osteoarthritis), a pain therapy (e.g., disc regeneration, Nerve inflammation, sacrum iliac joint, facet joint capsule), a vascular therapy (e.g., diabetic foot, peripheral arterial occlusion, bud formation of new arterioles), a uro-gynecology therapy (e.g., urinary incontinence, scleroatrophic lichen, anal fistulas), pressure ulcers and intestinal fistula. In one or more embodiments, the extracted purified adipose tissue is for a cosmetic or a medical use selected from the group consisting of a cosmetic treatment, an orthopedic treatment, a pain therapy, a vascular therapy, a uro gynecology therapy.

In one or more embodiments, the cosmetic indication includes using the processed tissue as a filler material.

### BRIEF DESCRIPTION OF THE DRAWINGS

The various features of the invention will best be appreciated by simultaneous reference to the description which follows and the accompanying drawings, which are not drawn to scale and in which:

FIG. 1 is an exemplary tissue processing system according to an aspect of the present invention.

FIG. 2 is an exemplary tissue processing chamber according to an aspect of the present invention.

FIG. 3 shows a flow chart of an exemplary method of priming the system according to an aspect of the present invention.

FIG. 4 shows a flow chart of an exemplary method of priming the tissue processing chamber according to an aspect of the present invention.

FIG. 5 shows an exemplary method of priming the system according to an aspect of the present invention.

FIG. 6 shows a flow chart of an exemplary method of washing an extracted tissue sample using an exemplary system according to an aspect of the present invention.

FIG. 7 shows an exemplary method of washing an extracted tissue sample using an exemplary system according to an aspect of the present invention.

FIG. 8 shows a flow chart of an exemplary method of filtering (e.g., micro-fragmenting) an extracted tissue sample using an exemplary system according to an aspect of the present invention.

FIG. 9 shows an exemplary method of filtering (e.g., micro-fragmenting) an extracted tissue sample using an exemplary system according to an aspect of the present invention.

FIG. 10 shows a flow chart of an exemplary method of filtering (e.g., nano-fragmenting) an extracted tissue sample using an exemplary system according to an aspect of the present invention.

FIG. 11 shows an exemplary method of filtering (e.g., nano-fragmenting) an extracted tissue sample using an exemplary system according to an aspect of the present invention.

FIG. 12 is an exploded view of the various elements of an exemplary tissue processing system according to an aspect of the present invention.

FIG. 13 is another exemplary tissue processing system according to an aspect of the present invention.

FIG. 14 show the expandable bag of the tissue processing system without (FIG. 14A) and when filled with (FIG. 4B) physiological fluid and adipose tissue.

FIG. 15 are micrographs illustrating the cell vitality of adipose tissues that have been processed by the herein invention (B and D) vs. original lipoaspirate tissues (A and C). The tissues have been harvested from human abdominal fat. A, C are representative images of two different original lipoaspirates harvested from two different subjects that didn't undergo any tissue processing; B is a representative image of tissue that has been processed by the herein invention, the tissue is obtained from the same subject of lipoaspirate shown in A; and D is a representative image of tissue that has been processed by the herein invention, the tissue is obtained from the same subject of lipoaspirate shown in C.

FIG. 16 are representative micrographs illustrating the structure of the stromal-vascular niche of a tissue that has been processed by the herein invention (LP) vs. an original lipoaspirate tissue (LA). The images show the texture of the stromal-vascular niche, as depicted by VE-Cadherin (green) and CD31 (red) staining.

FIG. 17 are whole-field view representative micrographs illustrating the stromal-vascular niche of a tissue that has been processed by the herein invention (LP) vs. an original lipoaspirate tissue (LA). These images show the texture of the stromal-vascular niche, as depicted by VE-Cadherin (green) and CD31 (red) staining.

FIG. 18 are representative micrographs illustrating Perilipin expression (green staining), a marker for the structural and functional integrity of prediapocytes and adipocytes, and the presence of CD68-positive cells (red staining), a marker for macrophages, major players in regenerative processes, in original lipoaspirate (LA) vs. a tissue that has been processed by the herein invention (LP).

### DETAILED DESCRIPTION

In one aspect the present invention is of a system for tissue processing. The tissue may be adipose tissue. In a further aspect, the present invention provides a method of processing a tissue, such as adipose tissue.

The system and methods of use thereof of the present invention have many advantages. The system provides a processed adipose tissue comprising adipose cells with a reduction in the size of the adipose cluster. The entire process is in a closed system in combination with a physiological solution, thereby eliminating contact with air and avoiding contamination of the adipose tissue, for optimal sterility. A cannula of liposuction comprising the adipose tissue extracted from a subject can be connected directly to the processing system, which allows the adipose tissue to be removed and transferred to the system without the tissue in contact with air, reducing the risk of contamination and oxidation. The system and method efficiently remove oils, membranes, cellular debris, blood cells, and inflammation components. The stromal vascular niche of the extracted adipose tissue is preserved. The system makes use of filters for reducing the adipose tissue cluster size. Stress on the cells and tissue is minimized by the procedure taking place in a physiological solution. Reduction of the adipose cell cluster results in an increase in the ratio between the surface and volume with a greater exposure of the vascular stromal niche providing a greater bioavailability in the release of cytokines and growth factors. Once the processed and purified tissue product is transplanted, it preserves the biological characteristics of the natural adipose connective tissue and can be injected with a needle and immediately be vascularized, acting as a natural scaffold for the cells by increasing their vitality. The system can be used for processing small and large quantities of adipose tissue. A further advantage of the tissue processing system of the present invention is that due to the total closed-circuit processing, the processed adipose tissue may be cryopreserved at authorized tissue banks for future use.

As used herein the terms ‘a’ and ‘an’ may mean ‘one’ or ‘more than one’.

As used herein the terms ‘comprising’, ‘including’, ‘containing’, ‘featuring’, ‘having’ and any forms of the terms thereof are inclusive and open ended and do not exclude additional elements or method steps, which are not recited.

The term ‘consisting essentially of’ as used herein means that the scope is limited to the specified elements and those that do not materially affect the basic and novel characteristic(s) of the claimed device and materials.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

All ranges disclosed herein include the endpoints. The use of the term “or” shall be construed to mean “and/or” unless the specific context indicates otherwise.

The term ‘about’, in some embodiments, refers to ±30% of the stated numerical value. In further embodiments, the term refers to ±20% of the stated numerical value. In yet further embodiments, the term refers to ±10% of the stated numerical value.

The principles and operation of a tissue processing system, as well as methods of use thereof according to the present invention may be better understood with reference to the figures. The figures show non-limiting aspects of the present invention.

The present invention provides a tissue processing system. The system may be used for processing adipose/fat tissue or lipoaspirate. The system may include a tissue processing chamber connected to a waste bag and a purging system.

As used herein the term “lipoaspirate” refers to a fat graft that has been harvested from a fat tissue of the body with, for example, a cannula.

As used herein the term “adipose tissue” refers to body fat, or fay tissue. Adipose tissue is a specialized connective tissue composed mostly of adipocytes, which are cells specialized for the storage of energy in the form of fat. Adipose tissue contains adipocytes, and stromal vascular fraction (SVF) of cells including preadipocytes, fibroblasts, mesenchymal stem cells, pericytes, vascular endothelial cells and a variety of immune cells such as macrophages, T cells and hematopoietic cells.

The tissue sample may be an adipose tissue taken from a subject by a needle or cannula aspiration. The tissue may be taken from various body locations. For example, the tissue can be taken from tissue beneath the skin (subcutaneous fat), around internal organs (visceral fat), intermuscular (Muscular system) and in the breast (breast tissue). Adipose tissue is found in specific locations, which are referred to as adipose depots.

The herein system is suitable to process a range of tissue volumes. For example, a volume of up to about 300 ml tissue may be processed without the need to divide the tissue sample. Suitable tissue sample volumes include up to about 300 ml, up to about 200 ml, up to about 150 ml, up to about 125 ml, up to about 100 ml, up to about 80 ml, up to about 60 ml, up to about 40 ml, up to about 20 ml, or up to about 10 ml of tissue to be processed. In exemplary embodiments, the system is suitable to process a tissue volume of about 48 ml, 60 ml, 120 ml, and 280 ml.

The herein system comprises a tissue processing chamber comprising an expandable bag that applies radial compression forces on the fluids inside it and by that may afford a controllable fluid flow rate out of the chamber.

The herein system may encompass one or more filters that afford filtration or fragmentation of the adipose tissue.

As used herein the term “micro fragmentation” refers to filtration or fragmentation of the tissue sample to the microscale, typically in the range of micrometers. This involves reducing the size of the tissue clusters to dimensions measured in micrometers. Suitable filters for micro fragmentation include, for example, filter with pores sizes of 3000 μm-70 μm.

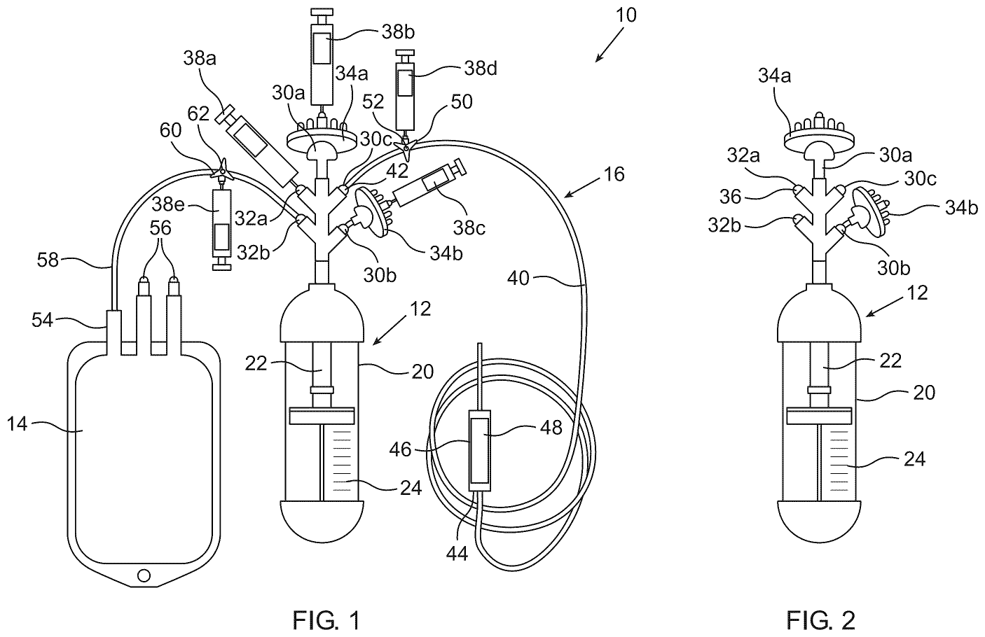

FIG. 1 shows an exemplary tissue processing system 10 according to an aspect of the present invention. The processing system 10 may include a tissue processing chamber 12 for separating, fragmenting, and/or cleaning components out of a tissue sample, such as a liposuctioned adipose tissue, a waste container, such as waste bag 14 for collecting waste material separated out of the liposuctioned adipose tissue sample and a purging apparatus 16 comprising physiological solution for washing the tissue.

The tissue processing chamber 12 is shown in FIG. 2. As can be seen from FIG. 2, the tissue processing chamber 12 may include a housing 20 accommodating an expandable bag 22 therein. The expandable bag 22 may be a dynamically fillable bag. The expandable bag 22 may be configured for holding the sample of adipose tissue and a volume of physiological fluid. The expandable bag 22 may be constructed from an elastic material, which can expand when fluid is introduced into the bag and contract when the fluid is removed from the bag. The expandable bag 22 may be an elastomeric balloon manufactured from rubber-like polymers with visco-elastic properties. The expandable bag 22 may be configured to release its contents using the passive return to the contracted state of the bag 22. The tissue processing chamber 12 may be graduated 24 and may include any suitable marking to indicate the volume of fluid in the expandable bag 22 for monitoring of the tissue purification process. The present invention may provide a tissue processing chamber 12 of any suitable size and volume. The size of tissue processing chamber 12 may be selected according to the volume of adipose tissue to be processed. Non-limiting examples of suitable volumes of tissue processing chamber 12 include 300 ml, 280 ml, 120 ml, 60 ml and 48 ml. For example, a volume of up to 300 ml, 250 ml, 200 ml, 150 ml, 100ml, or 50 ml may be processed and accommodated in the tissue processing chamber 12, and specifically in the expandable bag 22. For example, at least a volume of 50 ml, 100 ml, 150 ml, 200 ml, 250 ml, or 300 ml may be processed in the tissue processing chamber 12.

The tissue processing chamber 12 may be a closed device with at least one inlet 30 and at least one outlet 32 as shown in FIG. 2. In the exemplary system shown in FIG. 2, the tissue processing chamber includes branched inlets 30 and outlets 32. The figure shows an inlet 30c for introducing the tissue sample directly into tissue processing chamber 20. Inlet 30c may be used for introducing the physiological solution and/or for introducing the tissue sample. The figure shows an inlet 30a connected to a first filter 34 such as a micro filter 34a and an additional inlet 30b connected to a second filter 34b having pore sizes with a smaller diameter relative to first filter 34a. The tissue may be introduced to system 10 via any one of inlets 30a, 30b and 30c and/or filters 34a and 34b. Also shown in FIGS. 1 and 2 an outlet 32b is for collecting waste material and an outlet 32a is for collecting the processed tissue. Optionally, outlet 32b may be used as an inlet for introducing the tissue to be processed. The inlets 30 and/or outlets 32 may include a septum 36. The septum 36 may be made of any suitable material, such as, but not limited to rubber. The septum 36 may be penetrable by a syringe needle. A sample of, for example adipose tissue aspirate may be held in a syringe 38 and a user may push a connected needle to penetrate through the inlet seal 36. Syringe 38 may be held at this position and the syringe 38 contents may be expelled from the syringe 38 and introduced into the expandable bag 22 of the tissue processing chamber 12. In an alternative embodiment the inlets 30 and/or outlets 32 may feature a luer lock or a vakloc to facilitate a vacuum locking syringe mechanism and the tissue or physiological fluid may be introduced into the system 10 or removed from the system 10 without a needle.

The herein system 10 may comprise at least one filters 34 for reducing the size of the clusters of the tissue. The system 10 may comprise a plurality of filters 34. The system 10 may comprise two or more filters 34. In one embodiment, the filter 34 is disposed outside of the tissue processing chamber 12. In one or more embodiments, the filter 34 comprises a mesh being made from a polymeric material. The mesh is selectively permeable and allows reducing the size of the tissue clusters and provide tissue cluster reduction. The use of a polymeric material rather than a metal is advantageous, since it affords a gentler and tissue preservative procedure. In one or more embodiments, the filter comprises a mesh within a housing. In some embodiments, the system comprises or consists of one filter. In some embodiments, the system comprises or consists of two filters. In some embodiments, the system comprises or consists of three filters. In some embodiments, the system comprises or consists of four filters. In some embodiments, the system comprises or consists of five filters. In one or more embodiments, the mesh has a pore size of at least about 70 μm or 100 μm. For example, the mesh has a pore size of at least about 70 μm and up to about 2400 μm. For example, the mesh has a pore size of at least about 70 μm, 400 μm, at least about 500 μm, at least about 700 μm, or at least about 1200 μm. In one embodiment, the mesh has a pore size of about 70 μm. In one embodiment, the mesh has a pore size of about 200 μm. In one embodiment, the mesh has a pore size of about 400 μm. In one embodiment, the mesh has a pore size of about 700 μm. In one embodiment, the mesh has a pore size of about 1200 μm. In one embodiment, the system comprises two filters, wherein the first filter has mesh with a pore size of about 1200 82 m, and the second filter has mesh with a pore size of 700 μm. In one embodiment, the system consists of two filters, wherein the first filter has mesh with a pore size of about 1200 μm, and the second filter has mesh with a pore size of 700 μm. In one embodiment, the system comprises or consists of three filters, wherein the first filter has a mesh with a pore size of about 400 μm, the second filter has a mesh with a pore size of 200 μm and the third has pores size of 70 μm. In one embodiment, the system comprises or consists of five filters, wherein the first filter has a mesh with a pore size of about 1200 μm, the second filter has a mesh with a pore size of 700 μm, the third has a mesh with a pore size of 400 μm, the fourth has a mesh with a pore size of 200 μm, and the fifth has a mesh with a pore size of 70 μm.

Optionally, the mesh has a pore size of below 3000 μm. Fine filters having pore size of 3 or 2 microns or below, for example, 150 nm (nanometer) or 20-50 nm are also contemplated by the herein invention and allow filtering out oily components, and/or proteins (e.g., inflammatory agents), and/or debris from the adipose tissue. Such filtration affords a tissue sample product comprising also MUSE cells and/or exosomes.

The system 10 may include at least one inlet 30. The system 10 may include at least one outlet 32. In an exemplary embodiment, system 10 includes up to three inlets, as illustrated in FIGS. 1 and 2, i.e., inlets 30a, 30b, and 30c, and two outlets, i.e., outlets 32a, and 32b. First inlet 30c may be used for introducing a physiological fluid into the system or the tissue sample. Second inlet 30a may be connected to a first filter 34a for fragmentation of the sample into a first tissue fragments size. Third inlet 30b may be connected to a second filter 34b for smaller fragmentation. First outlet 32b may be used for waste material collection in the waste bag 14 and second outlet 32a may be for collecting the processed tissue. The tissue processing chamber 12 may include at least one inlet 30 connected to a filter 34. The filter 34 may be made from a plastic material. The fragmentation of adipose tissue into small clusters, such as about 700 microns, or about 1200 microns may ensure better survival of an adipose tissue implant and allow greater exposure and bio availability of the vascular-stromal niche. Different sized filters 34 may be employed for different sizes of fragmentation of the tissue sample. One inlet 30a may be connected to a microfilter 34a for micro fragmentation of the sample. The first filter 34a pore size may be about 1200 microns. One inlet 30b may be connected to a second filter 34b for smaller fragmentation.

The second filter 34b pore size may be about 700 microns. A tissue sample may be held in a syringe 38 and the syringe may be attached to the respective filter 34. The sample may then be expelled from the syringe 38 and filtered through the filter 34. The filtered sample may then be collected in the expandable bag 22 of the tissue processing chamber 12. In some embodiments, a tissue sample may be micro filtered, or nano filtered. In some embodiments, a tissue sample may be sequentially filtered by the first filter 34a and the second filter 34b, whereby the tissue sample may be micro fragmented by filtering through a micro filter 34a. The micro fragmented tissue may then be removed from the tissue processing chamber 12 using the syringe 38a. The syringe 38a with the micro fragmented tissue may then be attached to the second filter 34b and the micro fragmented tissue may be filtered for smaller fragmentation of the tissue. In some embodiments, the tissue sample may be sequentially filtered by filters with different mesh pore sizes. The sequential filtration may not be limited to filtration by micro filters and then nano filters. The tissue may be initially filtered with a first filter of larger pore size than a subsequent (second) filtration with a filter of smaller pore size.

Inlet 30c may be used for introducing a physiological fluid. The physiological fluid may be introduced into system 10 via a purging apparatus 16 as shown in FIG. 1. Purging apparatus 16 may include a channel 40, such as, but not limited to tubing. Channel 40 may be connected at a proximal extremity 42 to the tissue processing chamber inlet 30c. The channel 40 may be connected at a distal extremity 44 to a reservoir 46 of physiological fluid 48. Reservoir 46 may be a syringe 38 or may be any suitable container holding the physiological fluid 48. Non-limiting examples of physiological fluid 48 include saline, water, and phosphate buffered saline (PBS). Purging apparatus 16 may feature a multi-way valve 50. In one embodiment, the multi-way valve 50 may be a three-way valve. The multi-way valve 50 may be manipulated for allowing or stopping a physiological fluid 48 to be introduced from the reservoir 46 of physiological fluid, through the channel 40 and inlet 30c into the tissue processing chamber 12. The multi-way valve 50 may be manipulated to stop a physiological fluid 48 from entering system 12 from the physiological fluid reservoir 46. The multi-way valve may include a portal 52. The multi-way valve 50 may be manipulated via portal 52 to allow a syringe 38d connected to the portal 52 to expel its contents into the tissue processing chamber 12 through the corresponding inlet 30c in the tissue processing chamber 12.

The tissue processing chamber 12 is connected via an outlet 32b to a waste container which may be a waste bag 14. The waste bag 14 may include a waste bag inlet 54 and may include at least one waste bag outlets 56. The waste bag 14 may be connected to the tissue processing chamber 12 by a waste bag channel 58, such as for example suitable tubing. Channel 58 may include a multi-way valve 60. In one non-limiting example the multi-way valve 60 is a three-way valve. The valve 60 can be closed so that fluid is not expelled from the tissue processing chamber 12 into the waste bag 14. Valve 60 can be opened so that waste fluid can be expelled from the tissue processing chamber 12, through the waste bag channel 58 and into the waste bag 14 via the waste bag inlet 54. The multi-way valve 60 may have a portal 62 which may manipulate the valve 60, and which can be connected to a syringe 38c. The syringe 38e can be used to introduce a solution into the waste bag 14. The syringe 38e can also be used to collect waste expelled through the waste bag 14 via channel 58. The waste bag 14 may be made from any suitable material. The waste bag 14 may be of any suitable size and volume, e.g., up to 1-2 Liters. The waste bag 14 may be configured for collecting material separated from the tissue sample, such as, but not limited to inflammatory components, physiological solution, exosomes and other cellular fractions or debris. Components, such as exosomes and MUSE (multi-lineage differentiating stress enduring) cells collected in the waste bag 14 may be extracted via an outlet 56 of the bag 14. These biological components may have a plurality of medical applications. Optionally, the MUSE cells and/or exosomes are added to the purified adipose tissue after extraction thereof from the waste bag 14 (e.g., using dedicated filters). For example, it is possible to separate between MUSE cells and exosomes, using the appropriate filter having pore size of below about 15 μm-13 μm. Alternatively, it is possible to filter both MUSE cells and exosomes using larger pore sizes, such as filters of above 13˜15 μm. Advantageously, adding MUSE cells and/or exosomes to the purified adipose tissue, may contribute to the regenerative potential of the fat tissue. MUSE cells are embryonic-like stem cells expressing embryonic stem cells markers (e.g., SSEA3). These cells can differentiate into various cellular phenotypes, including, but not limited to, the cardiac, neural, skeletal muscle, and vascular lineages. Accordingly, adding MUSE cells could essentially contribute to the regenerative capacity of the purified adipose tissue processed by the herein system.

In an alternative embodiment, the MUSE cells and/or exosomes isolated from the waste bag 14 can be used individually, without the processed adipose tissue.

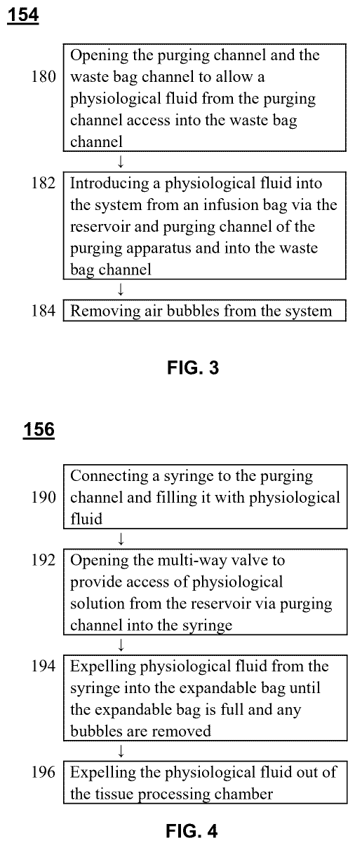

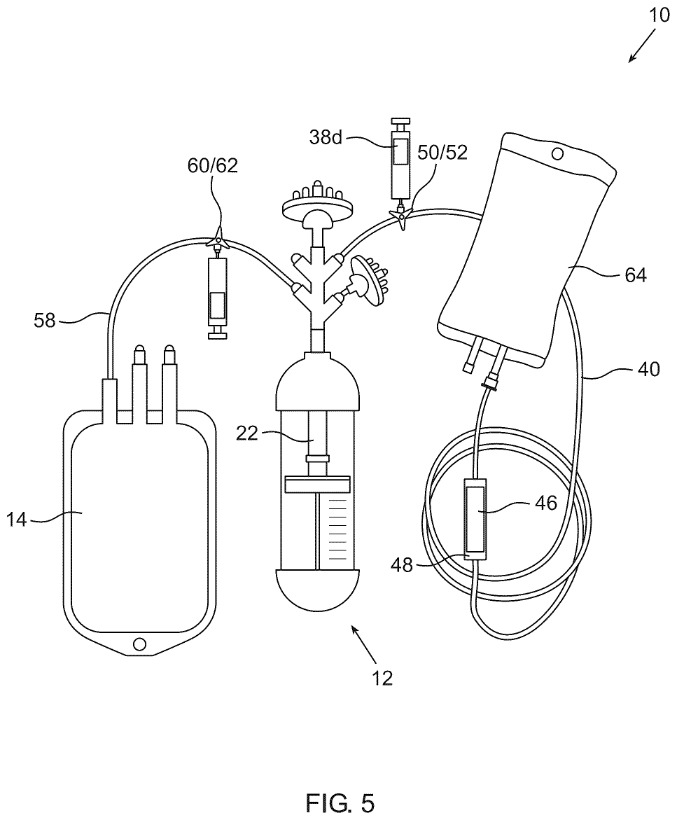

FIGS. 3-5 show exemplary methods 154 and 156 of priming the system. The order of the steps is not meant to be limiting and any suitable order may be used. The priming may feature washing the system 10 and removing air from the system 10. At step 180, the multi-way valves 50 and/or 60 of the purging channel 40 and/or waste channel 58, respectively, are opened so that a physiological fluid will flow from the reservoir 46 via purging channel 40 and into the waste channel 58 distal to the multi-way valve 60. The physiological fluid may flow into the waste bag 14. At step 182, a physiological fluid, such as, but not limited to saline is introduced into system 10. The physiological fluid can be injected through the purging apparatus 16 of the tissue processing system 10. The physiological fluid flows through the purging channel 40 of the purging apparatus 16 and into the waste bag channel 58. The system 10 has been primed when the air bubbles have been eliminated from the system, as illustrated at step 184.

The tissue processing chamber 12 may also be primed as illustrated in the flow chart 156 of FIG. 4. At step 190, a syringe 38d is connected to portal 52 and the multi-way valve 50 maneuvered. The multi way valve 50 may be firstly maneuvered to allow flow of physiological fluid derived from reservoir 46, so as to fill syringe 38d. Optionally syringe 38d is filled with physiological fluid by manually withdrawing fluid from the reservoir 46 into syringe 38d. At step 192, the multi-way valve 50 is then turned to open access from the syringe 38d into the tissue processing chamber 12. At step 194, physiological fluid from the syringe 38d is expelled into the expandable bag 22 of the tissue processing chamber 12 until the expandable bag 22 is full and any bubbles are expelled. The physiological fluid from the tissue processing chamber 12 may then be expelled as shown at step 196.

Priming may optionally include filling syringe 38d with physiological fluid by manually withdrawing fluid from the reservoir 46 into syringe 38d, opening valve 60 to thereby allow access of fluid from reservoir 46 and/or syringe 38d into waste bag 14, closing valve 60 and injecting fluid into tissue processing chamber 12.

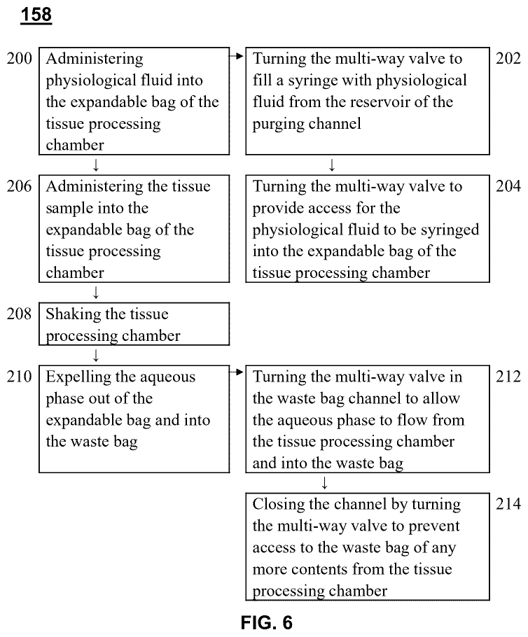

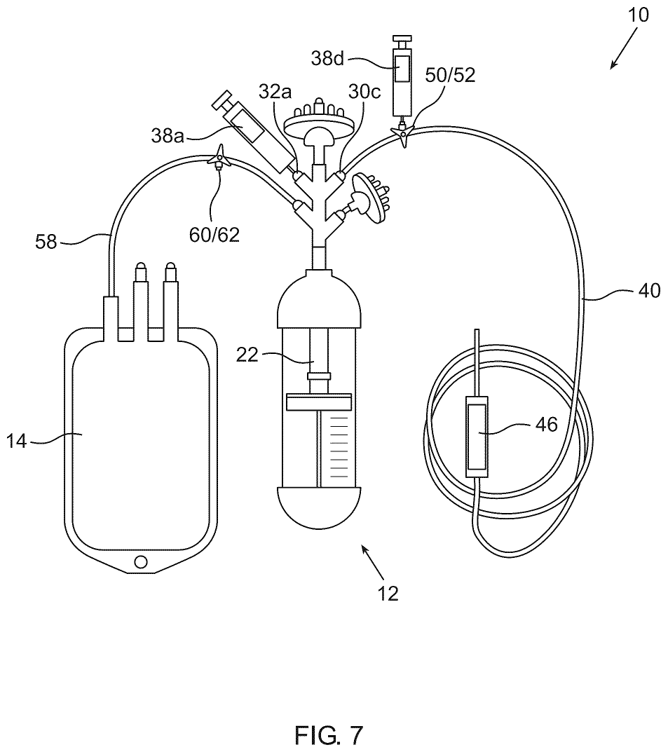

After priming the system 10, a lipoaspirated tissue may be washed by the herein system as depicted in the flow chart of FIG. 6 and in FIG. 7. At step 200, physiological fluid 48 within syringe 38d and/or reservoir 46 and/or infusion bag 64 may be introduced into the expandable bag 22 of the tissue processing chamber 12. Optionally, and as depicted at step 202 for filling the system 10 with physiological solution 48, a multi-way valve 50 is turned so that a syringe 38d connected to the multi-way valve 50 can fill up with physiological fluid 48 from the reservoir 46 and/or infusion bag 64 via purging channel 40. At step 204, the multi-way valve 50 is then turned to provide access to the physiological fluid in the syringe 38d to be syringed into the expandable bag 22 of the tissue 48 processing chamber 12. Alternatively, physiological fluid from reservoir 46 and/or infusion bag 64 may directly fill the expandable bag 22 after turning open the multi-way valve 50. Optionally, physiological fluid from syringe 38d may directly fill the expandable bag 22 by manually injecting the fluid into tissue processing chamber 12. The volume of physiological fluid may be determined by the volume of extracted tissue to process. About the same volume of physiological fluid as the volume of extracted tissue to be processed, may be introduced into the tissue processing chamber 12.

The adipose tissue to be processed may be a sample of tissue, which has been liposuctioned from a patient. The adipose tissue sample held in, for example a syringe 38a is administered into the expandable bag 22 of the tissue processing chamber 12, as depicted in step 206. In one embodiment, the syringe 38a is connected to the tissue sample outlet 32a which can be used, in some embodiments, as an inlet of the tissue processing chamber 12 of the processing system 10 and is injected therethrough into the expandable bag 22. The adipose tissue sample is received in the expandable bag 22. Optionally syringe 38d and inlet 30c can be used to administer the tissue sample to expandable bag 22.

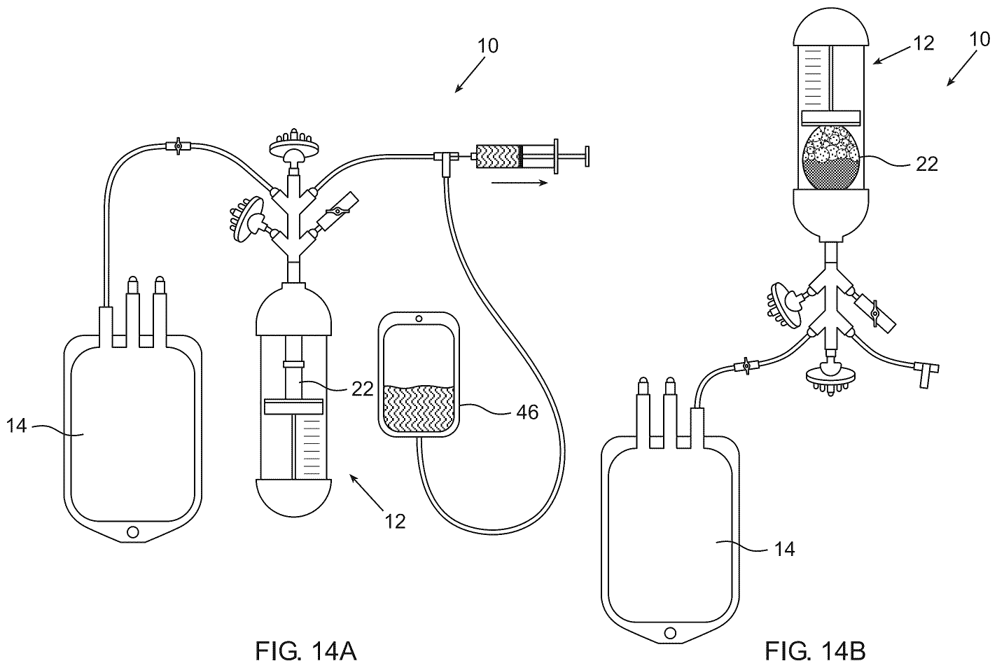

The adipose tissue sample may be washed without filtration. The adipose tissue sample may be washed without filtration as a first step. The adipose tissue sample may be washed with the physiological fluid contained in the expandable bag 22. At step 208, the tissue processing chamber 12 is shaken. The shaking can be done manually. In one embodiment, tissue processing chamber 12 can be mechanically shaken with a shaking device. The device is shaken and then kept still until two phases are seen. Optionally, system 10 or at least tissue processing chamber 12 is reversed and then shaken or shaken and then reversed. The fat phase will be the top phase, whilst below will lie the aqueous phase. The aqueous phase may contain debris, exosomes, cells and/or inflammatory agents separated from the adipose tissue sample. Upon filling the expandable bag 22 with the physiological fluid and/or adipose tissue, the expandable bag 22 may be expanded, as illustrated in FIG. 14B. Optionally, the purging channel 40 and reservoir 46 and/or infusion bag 64 are detached from the system 10 prior to adding the adipose tissue, or prior to shaking the tissue processing chamber 12. Optionally, system 10 or any portion thereof is turned upside down before the shaking step 208, such that the shaking step 208 is conducted when the system 10 or any portion thereof is in a reversed position as shown in FIG. 14B. Optionally, system 10 or any portion thereof is turned upside down after the shaking step 208.

At step 210, the remaining aqueous/lower phase may be expelled out of the expandable bag 22. The multi-way valve 60 in the waste channel 58 may be opened to allow the aqueous/lower phase to flow from the tissue processing chamber 12 and into the waste bag 14, as illustrated in step 212. When all the aqueous/lower phase has been removed from the tissue processing chamber 12, the multi-way valve 60 is turned to close the flow through channel 58 to waste bag 14 and prevent any more contents of the tissue processing chamber 12 from being expelled, as depicted at step 214.

The adipose tissue sample can be washed any suitable number of times. In one embodiment, the once washed adipose tissue sample is washed for a second time. The multi-way valve 50 may be turned so that a syringe 38d can fill up with a volume of physiological fluid from reservoir 46 via the purging channel 40. The multi-way valve 50 may then be turned to stop the syringe 38d filling up and provide access for the physiological fluid to be syringed into the expandable bag 22 of the tissue processing chamber 12. The volume of physiological fluid supplied to the expandable bag 22 may be about the same as the volume of extracted tissue to be processed. The physiological fluid is introduced into the expandable bag 22. The adipose tissue sample and the physiological fluid contained in the expandable bag 22 are shaken. The shaking may be manual. Maintaining chamber 12 still, after the shaking, results in two phases as shown in FIG. 14B. The aqueous phase may be expelled out of the expandable bag 22. The multi-way valve 60 in the waste bag channel 58 is opened by maneuvering portal 62 to allow access to the waste bag 14 and the aqueous phase is expelled from the tissue processing chamber 12 and into the waste bag 14. When all the aqueous phase has been removed from the tissue processing chamber 12, the multi-way valve 60 may be turned to close access to the waste channel 58 and prevent any more contents of the tissue processing chamber 12 from being expelled. The washed tissue, such as adipose tissue may be collected in a syringe 38a. A syringe may be connected to the outlet 32a of the tissue processing chamber 12 to collect all the washed tissue, such as fat held in the tissue processing chamber 12.

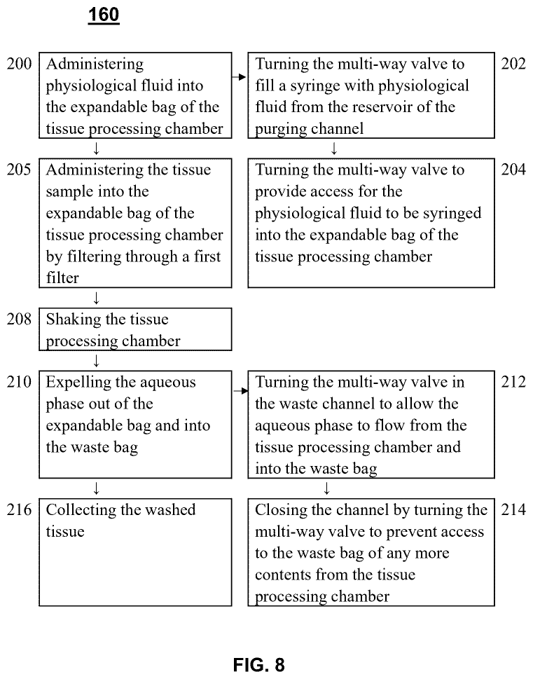

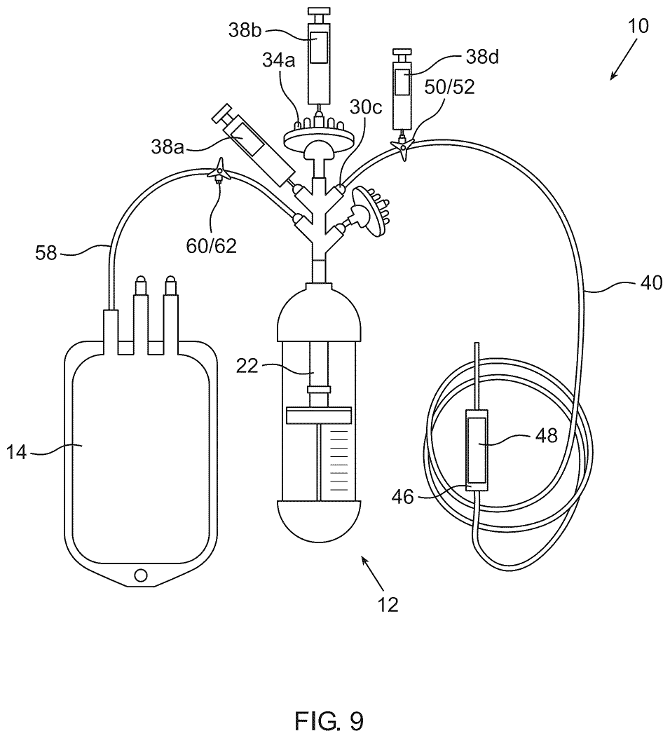

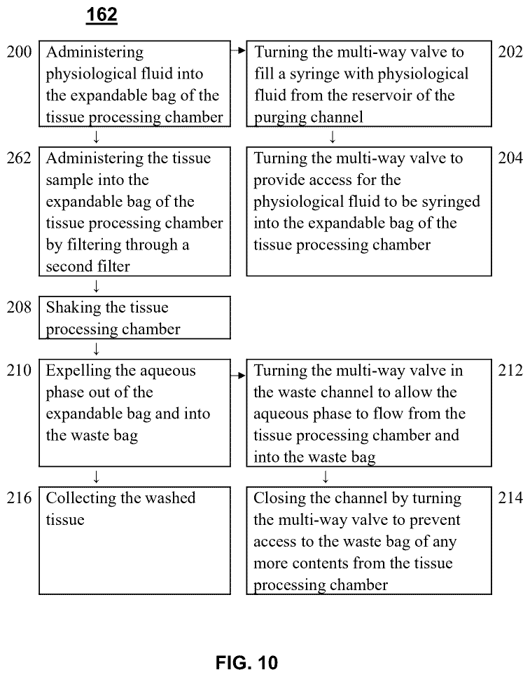

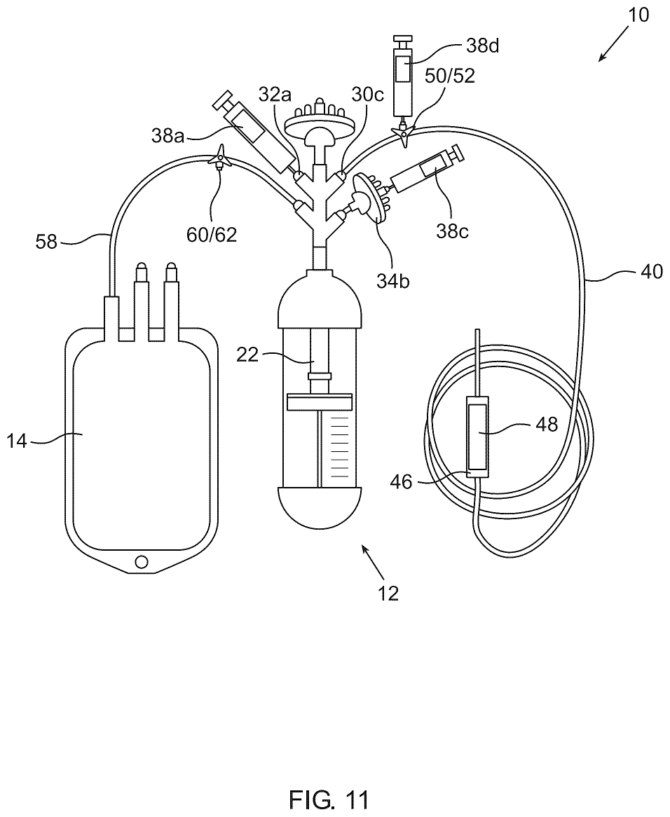

FIGS. 8-9 illustrate method steps 160 of fragmentation of an adipose tissue extracted from a subject. The adipose tissue may be micro fragmented. In some embodiments, the collected fat may be micro filtered and fragmented after the washing steps 158 as shown schematically in FIGS. 6-7. In some embodiments, the collected fat may be micro filtered directly without first conducting a washing step. Physiological fluid may be administered into the expandable bag 22 of the tissue processing chamber 12, by for example turning the multi-way valve to fill the syringe 38d with physiological fluid from reservoir 46 via the purging channel 40, as depicted in steps 200 and 202. The multi-valve 50 may be turned to provide the physiological fluid with access to the expandable bag 22 of the tissue processing chamber 12, at step 204. At step 205, the collected fat may be filtered through a first filter which may be a micro filter 34a connected to an inlet 30a of the tissue processing chamber 12 and the washing process, such as for example steps 208 to 214 described in FIGS. 6-7, may be repeated as above. In some embodiments, after micro filtering, the collected fat may then be filtered again using a second filter which may have smaller pores as shown schematically in FIGS. 10-11 depicting flowchart 162. In some embodiments, the biological sample may be filtered directly using second filter without preliminary using first filter. Physiological fluid may be administered into the expandable bag 22 of the tissue processing chamber 12 by for example turning the multi-way valve 50 to fill the syringe 38d with physiological fluid from the reservoir 46 via purging channel, as depicted in steps 200 and 202. The multi-valve 50 may be turned to provide the physiological fluid with access to the expandable bag 22 of the tissue processing chamber 12, at step 204. The collected processed fat or the biological tissue may be filtered through a second filter 34b connected to an inlet 30b of the tissue processing chamber 12, at step 262, and the washing process, such as for example steps 208 to 214 described in FIGS. 6-7 may be repeated as above.

The washed fat may then be collected in a syringe 38a, which may be suitably connected to an outlet 32a of the tissue processing chamber 12, at step 216. The collected fat is ready for reintroduction into the patient. The fat has been successfully purified and fragmented.

FIG. 12 is an exploded view showing the various elements of system 10 of the invention. System 10 includes a tissue processing chamber 12 that comprises an expandable bag 22 disposed within housing 20. The housing 20 may comprise a piston 23 which may be movable. Piston 23 surrounds a central rod 21. Rod 21 may extend along a longitudinal axis of the housing 20 or may extend down from piston 23. When the expandable bag 22 is filled with tissue sample and/or physiological fluid, the expandable bag 22 is expanded to various directions and the piston 23 may be pushed downwardly. Expandable bag 22 may be made from a polymer characterized by elastic properties, allowing it to return to its original shape after being stretched or deformed. The polymer exhibits a high degree of flexibility and resilience. Suitable types of polymers used to manufacture expandable bag 22 may include, for example, Natural Rubber (Polyisoprene), Styrene-Butadiene Rubber (SBR), Polybutadiene, Nitrile Rubber (NBR), Neoprene (Polychloroprene), Polyurethane, Silicone Rubber, and Ethylene-Propylene-Diene Monomer (EPDM). Each possibility represents a separate embodiment of the invention. In one or more embodiments, the polymers used to manufacture expandable bag 22 is selected from a rubber, Natural Rubber (Polyisoprene), Styrene-Butadiene Rubber (SBR), Polybutadiene, Nitrile Rubber (NBR),

Neoprene (Polychloroprene), Silicone Rubber, and Ethylene-Propylene-Diene Monomer (EPDM). Each possibility represents a separate embodiment of the invention.

Housing 20 may be made from a rigid polymeric material. Housing 20 may have various sizes. For example, housing 20 may be suitable to accommodate an expandable bag 22 capable of holding a fluid volume of up to about 300 ml, about 200 ml, about 100 ml, up to about 80 ml, up to about 60 ml, up to about 40 ml, up to about 20 ml, or up to about 10 ml. Each possibility represents a separate embodiment of the invention.

System 10 may include one or more filters 34 with various filter pores. The filters 34 include a filter housing 37 within which a mesh is disposed (not shown). Filter housing 37 comprises one or more entry ports 35 and an exit port 33. In one embodiment, the system 10 comprises or consists of one filter. In one embodiment, the system 10 comprises or consists of two filters. In one embodiment, the system 10 comprises or consists of three filters. In one embodiment, the system 10 comprises or consists of four filters. In one embodiment, the system 10 comprises or consists of five filters. Various sizes of the filter pores are contemplated, for example, 70 μm-2400 μm. In one embodiment, the system 10 comprises or consists of three filters, wherein the first filter has a mesh with a pore size of about 400 μm, the second filter has a mesh with a pore size of 200 μm and the third has pores size of 70 μm. In one embodiment, the system comprises or consists of five filters, wherein the first filter has a mesh with a pore size of about 1200 μm, the second filter has a mesh with a pore size of 700 μm, the third has a mesh with a pore size of 400 μm, the fourth has a mesh with a pore size of 200 μm, and the fifth has a mesh with a pore size of 70 umμm. In one embodiment, the system comprises or consists of two filters, wherein the first filter has a mesh with a pore size of about 1200 μm, and the second filter has a mesh with a pore size of 700 μm.

System 10 further includes a purging channel 40 connected to reservoir 46. Waste container, such as waste bag 14 is fluidly connected to main channel 59 via waste channel 58. Main channel 59 comprises a branched tubing comprising multiple tubing branches with entry/exit ports that can be connected to one or more filters. Various sizes of waste bag 14 and reservoir 46 are applicable. Waste bag 14 may be configured to hold a fluid volume of at least about 100 ml and up to about 2, or 3 Liters. Reservoir 46 may be configured to hold a fluid volume of at least about 10 ml and up to about 2, or 3 Liters. Reservoir 46 may feature a container which may be an infusion bag, a syringe, a bottle, or any other type of container that can hold fluids. Waste container 14 may feature a container which may be an infusion bag, a syringe, a bottle, or any other type of container that can hold fluids at a volume of at least about 10 ml and up to about 2, or 3 Liters.

All members in system 10 are fluidly connected, optionally via channels and tubing. For example, reservoir 46 may be connected to tissue processing chamber 12 via purging channel 40 which can connect main channel 59 via one or more inlets 30, or outlets 32, such as inlet 30c. Alternatively, reservoir 46 may be connected to tissue processing chamber 12 directly via a dedicated opening on tissue processing chamber 12. Waste bag 14 may be connected to tissue processing chamber 12 via waste channel 58 which can connect main channel 59 via one or more inlets 30, or outlets 32, for example outlet 32b. Optionally one or more elements in system 10 may be detached and then connected to the system 10. For example, main channel 59 with tubing branches 61 can be reversibly connected to housing 20. Similarly branches 61 can be connected and detached to main channel 59. One or more filters 34 can be reversibly connected to the one or more entry ports 30. One or both tubing 40 and 58 can be reversibly connected to main channel 59.

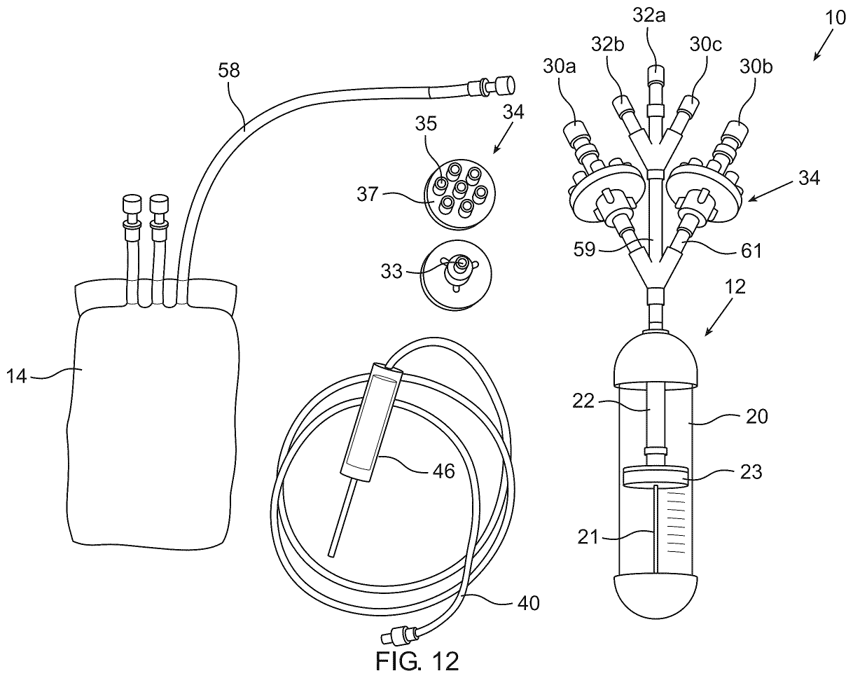

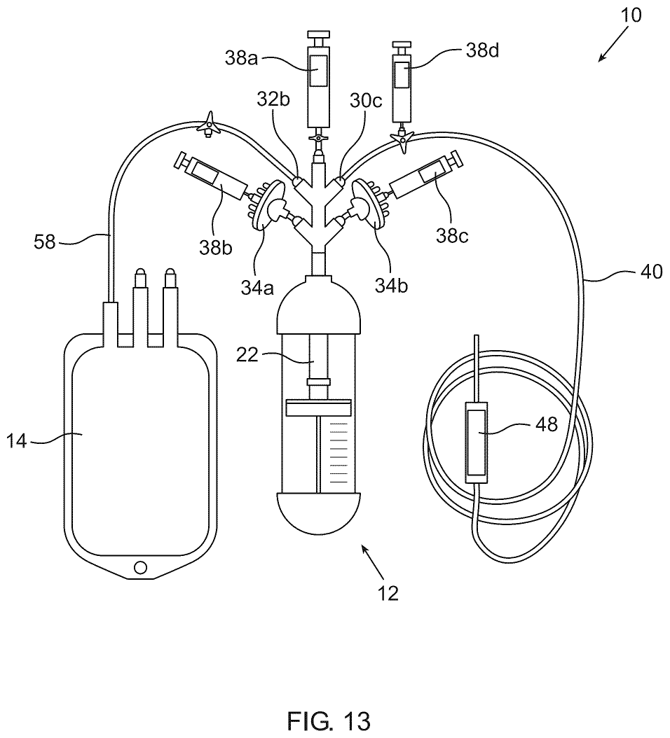

FIG. 13 illustrates system 10 wherein two symmetrical opposing inlets 30a and 30b are connected to first and second filters 34a and 34b. The first filter 34a may be of 1200 microns and the second filter 34b may be of 700 microns. Outlet 32 may be used to withdraw the tissue sample after processing thereof.

FIGS. 14A and 14B show the expandable bag of the tissue processing system without (FIG. 14A) and when filled with (FIG. 4B) physiological fluid and adipose tissue. The steps of tissue processing include withdrawing physiological solution from reservoir 46 with syringe 38d, closing valve 60, priming the tubing 40 and 58 of system 10, closing the valve 60, filling the chamber 12 with physiological solution, connecting syringe 38b and introducing the lipoaspirate through first filer 34a, reversing the system upside down, and shaking the chamber 12 to wash the tissue and purify it from inflammatory products. Following a few seconds-minutes, two phases are formed. Then the valve 60 of waste bag 14 is opened and the aqueous phase is withdrawn. To achieve the cleaned tissue a syringe 38a can be connected and the cleaned product withdrawn and may either be used or washed or filtered again.

Reference is made to the following examples, which together with the above descriptions illustrates the invention in a non-limiting fashion.

### EXAMPLES

### Example 1

A female subject wants to undergo a lip plumping treatment. A sample of adipose tissue is taken by liposuction from her stomach. The tissue is then processed prior to re-administration in her upper and lower lips. An adipose tissue processing system 10 of the present invention as described herein is used by the doctor to process the liposuctioned fat. He primes the system 10 with saline. Saline is introduced into the system 10 through the purging channel 40 until after the multi-way valve 60 of the waste channel 58. Air bubbles are removed from the system 10 in this way. The doctor primes the tissue processing chamber 12. Saline from the purging channel 40 is administered into the expandable bag 22 of the chamber 12. Air bubbles are removed from the chamber 12 and the bag 22 is emptied of the priming saline solution. The doctor then administers a volume of saline (20 ml) into the expandable bag 22, the volume equivalent to the volume of liposuctioned adipose tissue (20 ml) to be processed. The liposuctioned adipose tissue (20 ml) is introduced into the expandable bag 22 of the chamber 12. The doctor uses a syringe to administer the adipose tissue through a sample inlet in the system and into the bag 22 of the tissue processing chamber 12. The doctor shakes the bag 22, which holds the saline and adipose tissue sample. The tissue is washed and two layers form in the bag, a top fatty layer and a lower aqueous layer. The aqueous layer is cloudy. Impurities and unwanted water soluble components from the adipose tissue sample are washed into the saline, which results in the cloudy appearance of the aqueous layer. The doctor then releases the lower aqueous layer to remove it from the chamber 12 and into the waste bag 14. The doctor washes the adipose tissue for a second time. Saline (20 ml) is introduced into the expandable bag 22 of the tissue processing chamber 12. The chamber 12 is shaken. The aqueous layer is removed as described above. The doctor uses a syringe at the tissue inlet of the system to remove the cleaned adipose tissue from the bag 22 of the chamber 12. The doctor then micro fragments the washed tissue. He administers the washed adipose tissue through a micro filter 34a at a micro filter inlet 30a of the system 10 and into the expandable bag 22 of the chamber 12. The filtered adipose tissue is washed as described in the previous washing stage. The cleaned filtered adipose tissue is then removed from the chamber 12 with a syringe 38a via the tissue sample outlet 32a of the system. The processed adipose tissue is ready for reintroduction into the subject. The doctor injects 1 ml of the processed adipose tissue into the upper lip of the subject. The doctor injects 0.5 ml of the processed adipose tissue into the bottom lip of the subject.

### Example 2—Cells Viability

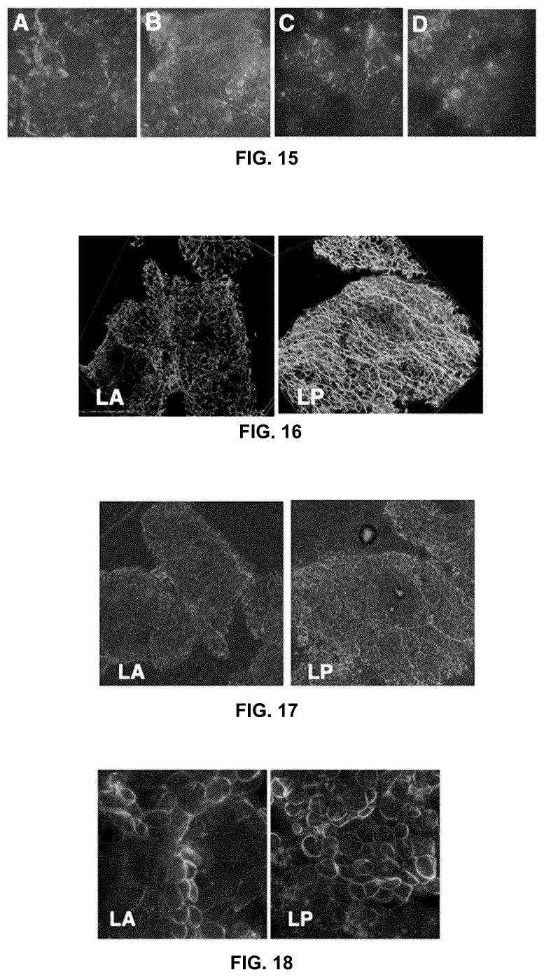

A cell vitality assay was used to evaluate cellular viability in tissue samples that have been processed by the herein device and method and in corresponding original lipoaspirate tissues (FIG. 15). The cell vitality assay is based on plasma membrane integrity and esterase activity. Cellular viability has been conducted using a two-color assay LIVE/DEAD™ Viability/Cytotoxicity Kit, for mammalian cells (Invitrogen™). FIG. 15 shows the presence of intact cellular contours (stained green), indicating a cell surface integrity. The number of cells exhibiting a red staining, resulting from the internalization of propidium iodide through a damaged plasma membrane is extremely low. Noteworthy, the number of red-stained cells is lower in the tissue that has been processed by the herein device and method (panels B and D), as compared with its corresponding original lipoaspirate (panels A and C), indicating that the fat processing achieved by the herein device was capable of eliminating distressed cells, thus increasing the overall yield of viable elements. Samples derived from tissue that has been processed by the herein device also showed consistent, well-evident alignments of green-stained, viable cells, which are referrable to as vascular structures within the sample itself (panels B and D).

### Example 3—Structural Analysis of the Stromal-Vascular Niche

Aliquots of samples of lipoaspirates and lipoaspirates that have been processed with the herein device and method were tested to evaluate the structure of the stromal-vascular niche. This is a “nano-topography”, where cells with pericyte identity (now emerging as the true precursors for mesenchymal stem cells) appear as perivascular elements within slit-like capillaries wedged between pre-adipocytes and stromal stalks. The stromal-vascular niche is a place where physical and chemical signaling coalesce to shape the biological features of tissue-resident stem cells, specifying both their differentiating and paracrine/autocrine repertoires.

The samples have been fixed and incubated with monoclonal antibodies targeted against VE-Cadherin (green staining) and CD31 (red staining) (FIG. 16). VE-Cadherin is an essential adhesion molecule in creating cell-to-cell physical and functional communications. It is also a remarkable component of the integrity and permeability of the vascular endothelium. CD31 (PECAM-1) is a pivotal marker for endothelial cells and pericytes, being also used to locate these cells and trace their distribution pattern within biological samples. Together, these markers allow a fine structural characterization of the stromal-vascular niche.

FIG. 16 illustrates representative samples of original lipoaspirate (LA), and of corresponding tissue that has undergo processing using the herein method and device (LP). These representative images show the remarkable texture of the stromal-vascular niche, as depicted by VE-Cadherin (green) and CD31 (red) staining. Intriguingly, the vascular architecture appeared more preserved in the LP sample, than in the original lipoaspirate (LA). This finding indicates that the herein device and method are capable of affording the recovery of an optimal architecture of the niche following the structural remodeling imposed by the stress of the liposuction procedure.

FIG. 17 shows a whole-field view of VE-Cadherin (green) and CD31 (red) staining in the original lipoaspirate (LA) and in its corresponding tissue that has been processed by the herein device and method (LP). The images show the stromal-vascular niche, and further confirm the above findings and conclusions. For the fluorescence analyses the same microscopy apparatus and set-up described above have been used. The images confirm the structural recovery and improvement in the architectural organization of the stromal-vascular niche in the LP, when compared to LA. The profile of the red staining for CD-31 is very suggestive, highlighting how the pericyte elements (perivascular located) follow the vascular network and intertwine with its path.

### Example 4—The Distribution Pattern of Perilipin and CD68

In additional characterization analyses, the distribution pattern of perilipin and CD68 in both lipoaspirate samples, and their corresponding tissues that have been processed by the herein device and method have been evaluated (FIG. 18). Perilipin (green staining) is a marker for the structural and functional integrity of prediapocytes and adipocytes, two important cellular elements in the modulation of the paracrine features of the stromal-vascular niche. CD68 (red staining) is a marker for macrophages, a cell type that is increasingly emerging as a major player in regenerative processes. After tissue injury, these cells undergo marked phenotypic and functional changes to play critical roles during the initiation, maintenance, and resolution phases of tissue repair. The LP tissues exhibited clearer/sharper contour in the membranes of pre-adipocytic elements (green), and macrophages (red), with a more ordered architectural organization of the cells as compared to the original lipoaspirate (LA). Consonant with the images in FIG. 15, these observations again suggest that the tissue processing using the herein device and method prompted a significant recovery in essential cellular and architectural features of the processed samples.

### Conclusions

The above examples illustrate that the herein device and method represents a new paradigm in the processing of adipose tissue, with unprecedented perspectives for the most varied applications in Regenerative Medicine, as well as Aesthetic and Reconstructive Medicine.

One skilled in the art can appreciate from the foregoing description that the broad systems, devices, and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1.-34. (canceled)

35. A tissue processing system for processing an extracted adipose tissue, comprising:
a tissue processing chamber comprising an expandable bag and configured for holding fluid and the adipose tissue, the tissue processing chamber is further configured for cleaning, separation, and/or fragmentation of components in the extracted tissue, the expandable bag is constructed from an elastic material, such that the bag expands when fluid is introduced into the bag and passively contracts when the fluid is removed from the bag, the expandable bag applies radial compression forces on the fluids inside thereby affording a controllable fluid flow rate out of the chamber,
at least one outlet for collecting waste and processed tissue;
at least one inlet for administering an extracted biological tissue into the tissue processing chamber; and
a waste container for collection of water-soluble components separated from the extracted tissue.

36. The system of claim 35, wherein the expandable bag is disposed within a housing that comprises a movable piston that surrounds a central rod, wherein when the expandable bag is filled with tissue sample and/or physiological fluid, the expandable bag is expanded to various directions, and the piston is pushed downwardly.

37. The system of claim 35, further comprising at least one channel for providing a physiological solution to the system and wherein the channel is optionally opened and closed with a multi way valve, and wherein the channel comprises tubing connected to an inlet of the system.

38. The system of claim 35, further comprising at least one filter that has a pore size of about 1200 microns (μm), or below, the filter connected to an inlet of the at least one inlet, configured to allow administration of the extracted tissue into the system via the at least one filter.

39. The system of claim 38, wherein the at least one filter has a pore size of about 700 microns (μm), or below.

40. The system of claim 35, wherein the waste container is connected to an outlet of the at least one outlet of the tissue processing chamber via a channel and wherein the channel is optionally opened and closed with a multi way valve, and wherein the waste container is a waste bag comprising at least one outlet port for extracting at least one component held in the waste bag.

41. The system of claim 35, wherein the expandable bag is configured for holding adipose tissue and an aqueous physiological solution.

42. The system of claim 35, wherein an outlet of the at least one outlet is connected to a syringe port for extraction of the processed tissue.

43. The system of claim 35, wherein the expandable bag is manufactured from a rubber material having visco-elastic properties, or from a polymer characterized by elastic properties.

44. The system of claim 35, wherein the system does not include a centrifuge, or an electric pump and/or does not require the use of an enzyme for processing the tissue.

45. A method of processing a tissue comprising:
providing a tissue processing system;
administering a physiological aqueous solution into an expandable bag of a tissue processing chamber within the tissue processing system, the expandable bag is constructed from an elastic material, such that the bag expands when fluid is introduced into the bag and passively contracts when the fluid is removed from the bag;
administering adipose tissue extracted from a subject into the tissue processing chamber, the tissue administered via an inlet of the system into the expandable bag;
shaking the contents of the tissue processing chamber;
removing the aqueous phase from the tissue processing chamber into a waste container; and
removing the purified adipose tissue out of the tissue processing chamber and out of the tissue processing system, wherein the administering and removing steps are conducted manually.

46. The method of claim 45, wherein prior to administering the adipose tissue into the system, the method comprises purging the system with a physiological aqueous solution to thereby remove air bubbles, wherein the system comprises at least one channel for providing a physiological solution to the tissue processing chamber, wherein access of the channel to the tissue processing chamber is opened and closed with a multi way valve, and wherein the purging comprises opening the multi way valve to provide the physiological solution into the tissue processing chamber.

47. The method of claim 45, wherein the administering adipose tissue into the system comprises administering the adipose tissue through a filter that has a pore size of between about 70 microns (μm) and up to about 3000 microns (μm) connected to the system, the filter disposed prior to the inlet of the tissue processing chamber and the method further comprises filtering the adipose tissue.

48. The method of claim 45, comprising
administering a physiological aqueous solution into the expandable bag of the tissue processing chamber;
administering the adipose tissue through a first filter connected to the system, the first filter is disposed prior to the inlet of the tissue processing chamber and filtering the adipose tissue into the expandable bag;
reversing the tissue processing chamber;
shaking the contents of the tissue processing chamber;
removing the aqueous phase from the tissue processing chamber into the waste container; and
removing the purified adipose tissue out of the tissue processing chamber and out of the processing system;
administering a physiological aqueous solution into the expandable bag of the tissue processing chamber;
administering the adipose tissue through a second filter connected to the system, the second filter having a pore size that is smaller than the pore size of the first filter, the second filter is disposed prior to the inlet of the tissue processing chamber and filtering the adipose tissue into the expandable bag;
reversing the tissue processing chamber;
shaking the contents of the tissue processing chamber;
removing the aqueous phase from the tissue processing chamber into the waste container; and
removing the purified adipose tissue out of the tissue processing chamber and out of the processing system.

49. The method of claim 45, wherein the administering an aqueous solution comprises administering about the same volume of aqueous solution as the volume of extracted tissue administered to the tissue processing chamber.

50. The method of claim 45, wherein the shaking is manually shaking for solubilizing water soluble components in the aqueous phase, wherein after shaking and stabilizing the tissue processing chamber, a two-phase solution is formed.

51. The method of claim 45, wherein the extracted purified adipose tissue is for a cosmetic or a medical use selected from the group consisting of a cosmetic treatment, an orthopedic treatment, a pain therapy, a vascular therapy, a uro gynecology therapy

52. The method of claim 45, wherein the aqueous phase comprises exosomes, inflammatory agents, and muse cells, and wherein the method further comprises separating the exosomes and/or muse cells from the waste container and collecting them.

53. The method of claim 45. further comprising cooling down the processed tissue to a temperature of less than 4° C. prior to injection thereof to the subject.

54. A tissue sample prepared by the method according to claim 45.
